(19) United States
(12) Patent Application Publication
(10) Pub. No.: US 2017/0209124 A1
(43) Pub. Date: Jul. 27, 2017

(54) Ultrasound Probe and Ultrasound Diagnostic Apparatus

(71) Applicant: KONICA MINOLTA, INC., Chiyoda-ku (JP)

(72) Inventors: Miyuki GAWAZAWA, Kanagawa (JP); Morio NISHIGAKI, Kanagawa (JP); Toshiharu SATO, Tokyo (JP)

(21) Appl. No.: 15/411,031

(22) Filed: Jan. 20, 2017

(30) Foreign Application Priority Data
Jan. 21, 2016 (JP) 2016-009884

(51) Int. Cl. A61B 8/00; G01N 29/22; A61B 8/08

(52) U.S. Cl. CPC A61B 8/4494; A61B 8/461; A61B 8/54; A61B 8/0841; A61B 8/4405; G01N 29/221; G01N 2291/02475

(57) ABSTRACT

An ultrasound probe includes transmission and reception sections, an acoustic lens, and a switch section. The transmission and reception sections include first, second, and third transmission and reception sections, while the second transmission and reception section is centrally positioned, and the first and the third transmission and reception sections are disposed symmetrically at both sides of the second transmission and reception section. The acoustic lens includes first, second, and third lens portions respectively corresponding to the first, the second, and the third transmission and reception sections. The switch section causes, when a traveling direction of the ultrasound is straight ahead, for example, the second transmission and reception section alone to operate, and causes, when the traveling direction of the ultrasound is to be deflected, the first or the third transmission and reception section to operate. The first and the third lens sections have an aspherical shape.

### CROSS REFERENCE TO RELATED APPLICATIONS

This application is entitled to and claims the benefit of Japanese Patent Application No. 2016-009884, filed on Jan. 21, 2016, the disclosure of which including the specification, drawings and abstract is incorporated herein by reference in its entirety.

### BACKGROUND OF THE INVENTION

1. Field of the Invention

The present invention relates to an ultrasound probe and an ultrasound diagnostic apparatus.

2. Description of Related Art

Ultrasound diagnostic apparatuses have been known, each conducting an examination of an internal structure of a test subject by irradiating the inside of the test subject with ultrasound, receiving a reflection wave (echo) of this ultrasound and performing predetermined signal data processing. Such an ultrasound diagnostic apparatus has been widely used for various applications including medical examinations and treatments, for example.

The ultrasound diagnostic apparatus not only displays an image after processing the acquired reflection wave data, but also uses an ultrasound image when a puncture needle is inserted to a target position while the positions of the puncture needle and the target are visually observed for taking, for example, a sample of a specific region (target) within the test subject, discharging water, or injecting and leaving medication or marker in a specific region. Moreover, an ultrasound image is used, for example, when a catheter is inserted into a specific region such as a bile duct while the positions of the catheter and the specific region are visually recognized. Such a use of an ultrasound image makes it possible for treatment of a target in the test subject to be conducted promptly, surely, and easily.

Among the ultrasound diagnostic apparatuses, an apparatus in which oscillators for transmitting and receiving ultrasound are arranged and which performs capturing while scanning (particularly, electronic scanning), in a predetermined arrangement direction, positions where transmission and reception of ultrasound is performed. For example, the puncture needle is inserted along this scanning direction and thus continuously positioned within a range where capturing is possible (hereinafter, referred to as “capturing range”) during an interval from the insertion position to the test subject to the point where the puncture needle arrives at the target.

However, the puncture needle does not necessarily travel accurately in the original insertion direction because of an internal condition and/or a structure of the test subject and/or the shape of a distal end of the puncture needle or the like, and/or the puncture needle is bent in some cases. As a result, there arises a problem in that the puncture needle is no longer captured because the distal end of the puncture needle moves out of the capturing range in a width direction orthogonal to the scanning direction.

To solve this problem, Japanese Patent Application Laid-Open No. 2000-139926 discloses a technique to perform capturing at an outer side of the original ultrasound transmission and reception width by providing delay circuits for delaying operation timings of a plurality of oscillators arranged in a width direction and by switching a magnitude relationship among the plurality of oscillators to deflect the traveling direction of ultrasound.

However, a deflection control circuit including these delay circuits for adjusting the capturing range increases in size and generates heat during its operation, thus involving a problem in that the usability of the ultrasound probe is reduced.

### SUMMARY OF THE INVENTION

It is therefore an object of the present invention to provide an ultrasound probe and an ultrasound diagnostic apparatus each allowing adjustment of a capturing range with a simpler configuration and without a reduction in usability.

To achieve at least one of the abovementioned objects, an ultrasound probe reflecting one aspect of the present invention includes: a plurality of transmission and reception sections arranged along a predetermined first direction and configured to transmit ultrasound to a test subject and receive a reflection wave of the ultrasound; an acoustic lens configured to focus, in the first direction, ultrasound beams transmitted and received by the transmission and reception sections; and a switch section configured to switch between operation and non-operation of the transmission and reception sections, wherein: the transmission and reception sections include first, second, and third transmission and reception sections, the second transmission and reception section being centrally positioned, the first and the third transmission and reception sections being disposed symmetrically at both sides of the second transmission and reception section, the acoustic lens includes first, second, and third lens portions respectively corresponding to the first, the second, and the third transmission and reception sections, the switch section causes, when a traveling direction of the ultrasound is straight ahead, the second transmission and reception section alone or all of the first, the second, and the third transmission and reception sections to operate, and causes, when the traveling direction of the ultrasound is to be deflected, the first or the third transmission and reception section to operate, and the first and the third lens portions have an aspherical shape.

Desirably, in the ultrasound probe, a split ratio of the first, the second, and the third transmission and reception sections in the first direction is 1:1:1.

Desirably, in the ultrasound probe, in the first and the third lens portions, a surface where the ultrasound is transmitted and received is entirely aspherical.

Desirably, in the ultrasound probe, a curvature of the aspherical shape becomes closer to a curvature of the second lens portion as the aspherical shape extends to the second lens portion from each end of the aspherical shape in the first direction of the first and the third lens portions.

To achieve at least one of the abovementioned objects, an ultrasound probe reflecting one aspect of the present invention includes: a plurality of transmission and reception sections arranged along a predetermined first direction and configured to transmit ultrasound to a test subject and receive a reflection wave of the ultrasound; an acoustic lens configured to focus, in the first direction, ultrasound beams transmitted and received by the transmission and reception sections; and a switch section configured to switch between operation and non-operation of the transmission and reception sections, wherein: the transmission and reception sections include first, second, and third transmission and reception sections, the second transmission and reception section being centrally positioned, the first and the third transmission and reception sections being disposed symmetrically at both sides of the second transmission and reception section, and the switch section includes first, second, and third switch sections respectively corresponding to the first, the second, and the third transmission and reception sections, wherein the second switch section includes a switching element and an electric circuit connected in parallel with the switching element, and causes the second transmission and reception section to operate via the electric circuit or not via the electric circuit by the switching element.

Desirably, in the ultrasound probe, a split ratio of the first, the second, and the third transmission and reception sections in the first direction is 1:2:1.

Desirably, in the ultrasound probe, the electric circuit reduces sensitivity of the second transmission and reception section of a case where the second transmission and reception section is caused to operate via the electric circuit compared to a case where the second transmission and reception section is caused to operate not via the electric circuit.

Desirably, in the ultrasound probe, the electric circuit includes a phase shift circuit.

To achieve at least one of the abovementioned objects, an ultrasound probe reflecting one aspect of the present invention includes: a plurality of transmission and reception sections arranged along a predetermined first direction and configured to transmit ultrasound to a test subject and receive a reflection wave of the ultrasound; an acoustic lens configured to focus, in the first direction, ultrasound beams transmitted and received by the transmission and reception sections; and a switch section configured to switch between operation and non-operation of the transmission and reception sections, wherein the transmission and reception sections include first, second, and third transmission and reception sections, the second transmission and reception section being centrally positioned, the first and the third transmission and reception sections being disposed symmetrically at both sides of the second transmission and reception section, wherein the second transmission and reception section includes first and second sections obtained by centrally splitting the second transmission and reception section, and the switch section includes switching elements corresponding to the first and the second sections, wherein the switch section causes, when a traveling direction of the ultrasound is straight ahead, the second transmission and reception section alone or all of the first, the second, and the third transmission and reception sections to drive, and causes, when the traveling direction of the ultrasound is to be deflected, one of the first and the second sections of the second transmission and reception section to drive by the corresponding switching element.

Desirably, in the ultrasound probe, a split ratio of the first, the second, and the third transmission and reception sections in the first direction is 1:2:1.

Desirably, in the ultrasound probe, the switch section includes a first switch section connected in common to the first and the third transmission and reception sections.

To achieve at least one of the abovementioned objects, an ultrasound diagnostic apparatus reflecting one aspect of the present invention includes: the ultrasound probe; and a transmission and reception processing section configured to perform a transmission and reception operation of ultrasound in the ultrasound probe.

### BRIEF DESCRIPTION OF DRAWINGS

The present invention will become more fully understood from the detailed description given hereinbelow and the appended drawings which are given by way of illustration only, and thus are not intended as a definition of the limits of the present invention, and wherein:

FIG. 1 is a diagram illustrating an overall configuration of an ultrasound diagnostic apparatus according to a first embodiment of the present invention;

FIG. 2 is a block diagram illustrating an example of an internal configuration of the ultrasound diagnostic apparatus;

FIG. 3 is a diagram illustrating an example of transmission and reception arrangement of an ultrasound probe in a short-axis direction;

FIG. 4A is a diagram illustrating a relationship between a center part transmission and reception section to be used and a shape of an ultrasound beam;

FIG. 4B is a diagram illustrating a relationship between all transmission and reception sections to be used and a shape of an ultrasound beam;

FIG. 4C is a diagram illustrating a shape of a combined ultrasound beam;

FIG. 5A is a diagram illustrating a relationship between a center part transmission and reception section to be used and a shape of an ultrasound beam;

FIG. 5B is a diagram illustrating a relationship between all transmission and reception sections to be used and a shape of an ultrasound beam;

FIG. 5C is a diagram illustrating a shape of a combined ultrasound beam;

FIG. 6 is a diagram illustrating a cross-sectional structure along a short-axis direction of transmission and reception arrangement in an ultrasound probe according to a comparison example;

FIG. 7 is a diagram illustrating a cross-sectional structure along a short-axis direction of transmission and reception arrangement in an ultrasound probe;

FIG. 8A is a diagram illustrating a relationship between a center part transmission and reception section to be used and a shape of an ultrasound beam;

FIG. 8B is a diagram illustrating a relationship between all transmission and reception sections to be used and a shape of an ultrasound beam;

FIG. 8C is a diagram illustrating a shape of a combined ultrasound beam;

FIG. 9 is a diagram illustrating an ultrasound probe according to a comparison example;

FIG. 10 is a diagram illustrating a relationship between a transmission and reception section to be used and a shape of an ultrasound beam;

FIG. 11 is a diagram illustrating an ultrasound probe according to a second embodiment;

FIG. 12A is a diagram illustrating an exemplary configuration of an electric circuit;

FIG. 12B is a diagram illustrating another exemplary configuration of the electric circuit;

FIG. 12C is a diagram illustrating another exemplary configuration of the electric circuit;

FIG. 13 is a diagram illustrating an ultrasound probe according to a third embodiment;

FIG. 14 is a diagram illustrating a relationship between a transmission and reception section to be used and a traveling direction of ultrasound; and

FIG. 15 is a diagram illustrating a relationship between a transmission and reception section to be used and a traveling direction of ultrasound.

### DESCRIPTION OF THE PREFERRED EMBODIMENTS

Hereinafter, a description will be given of embodiments of the present invention with reference to the drawings.

### First Embodiment

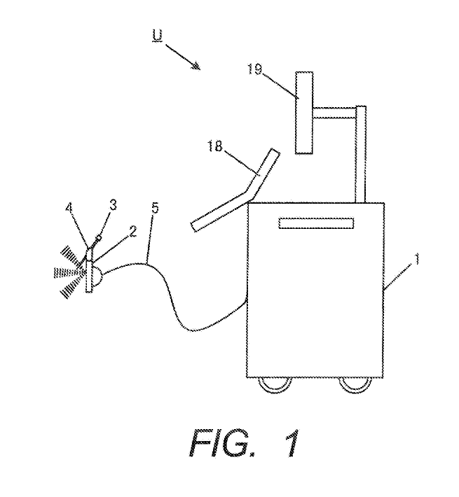

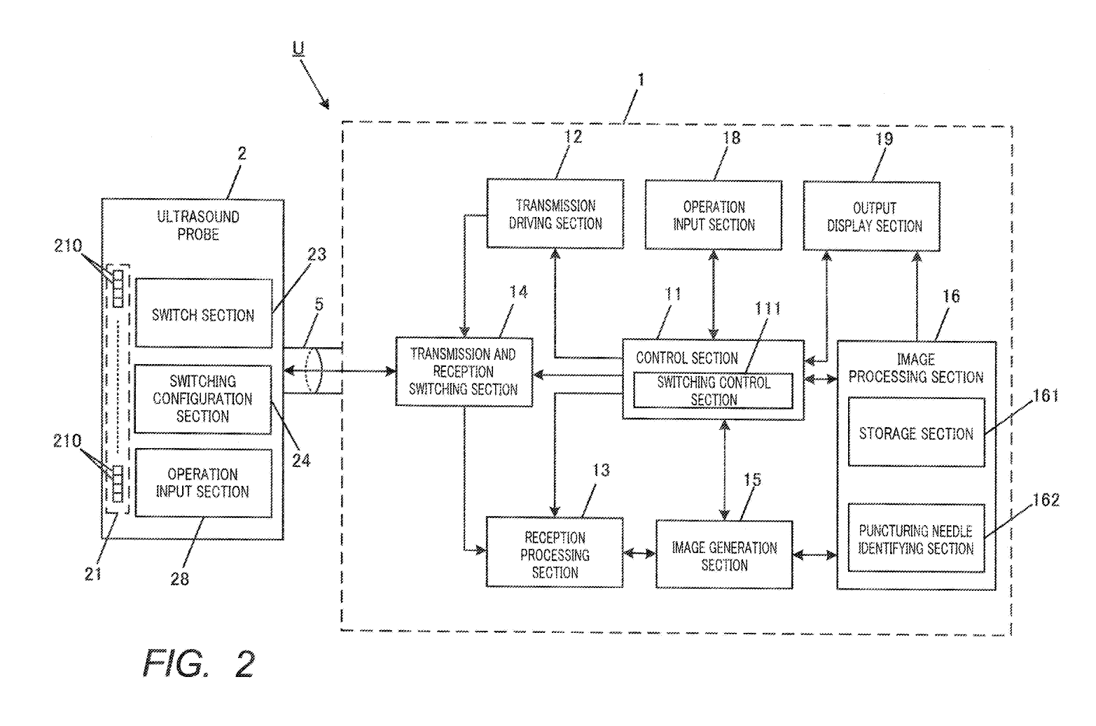

FIG. 1 is a diagram illustrating an overall configuration of ultrasound diagnostic apparatus U of the first embodiment. FIG. 2 is a block diagram illustrating an internal configuration of ultrasound diagnostic apparatus U.

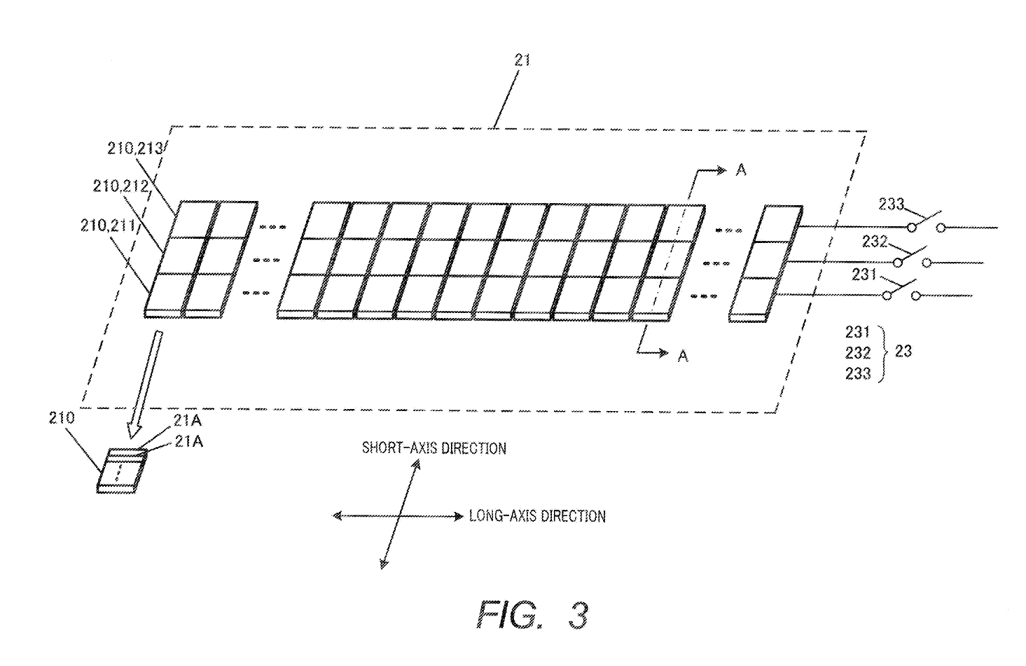

As illustrated in FIGS. 1 and 2, ultrasound diagnostic apparatus U includes: ultrasound diagnostic apparatus body 1; ultrasound probe 2 connected to ultrasound diagnostic apparatus body 1 via cable 5; puncturing needle 3; and attachment section 4 attached to ultrasound probe 2, for example. Note that, ultrasound probe 2 cited herein as an example is a so-called 1.25D probe including: three transmission and reception sections 210 arranged in a short-axis direction; and switch section 23 for switching between all of three transmission and reception sections 210 and some of three transmission and reception sections 210 as a driving transmission and reception section configured to perform transmission and reception of ultrasound. Moreover, regarding use of an ultrasound image, a description will be given of an example in which an ultrasound image is used in a case where puncturing needle 3 is inserted while positions of puncturing needle 3 and a target in a test subject are visually observed. However, the present invention is not limited to this example. Note that, transmission and reception section 210 has one or more oscillators 21A (see FIG. 3). In addition, each oscillator 21A included in each of first, second, and third transmission and reception sections 211, 212 and 213 arranged in the short-axis direction mutually performs transmission and reception simultaneously. Moreover, the number “three,” which is the number of transmission and reception sections 210, refers to the number for a case where a plurality of oscillators 21A arranged in the short-axis direction are sectioned into three parts including a center part and side parts positioned at both sides of the center part in the short-axis direction.

Puncturing needle 3 has a hollow long needle shape and is to be inserted into a test subject at an angle predetermined by a setting of attachment section 4. Puncturing needle 3 can be exchanged with one having an appropriate thickness, length and/or an appropriate leading end shape in accordance with a collection target (test body) or a type or an amount of medication or the like to be injected.

Attachment section 4 holds puncturing needle 3 in a set orientation (direction). Attachment section 4 is attached to a side part of ultrasound probe 2 and allows for changing and setting as appropriate the orientation of puncturing needle 3 in accordance with the insertion angle of puncturing needle 3 with respect to a test subject. Attachment section 4 allows not only for simply moving puncturing needle 3 in the insertion direction, but also for inserting puncturing needle 3 while rotating (spinning) puncturing needle 3 about a center axis of puncturing needle 3. Note that, a guide section configured to hold puncturing needle 3 in an insertion direction may be provided directly to ultrasound probe 2, instead of attachment section 4.

Ultrasound diagnostic apparatus body 1 is provided with operation input section 18 and output display section 19. As illustrated in FIG. 2, in addition to these components, ultrasound diagnostic apparatus body 1 includes control section 11, transmission driving section 12, reception processing section 13, transmission and reception switching section 14, image generation section 15, and image processing section 16, for example. Control section 11 of ultrasound diagnostic apparatus body 1 outputs a drive signal to ultrasound probe 2 to cause ultrasound probe 2 to output ultrasound, based on an external input operation on an input device such as a keyboard or mouse of operation input section 18. In addition, control section 11 of ultrasound diagnostic apparatus body 1 receives a received signal according to reception of ultrasound from ultrasound probe 2 and performs each type of processing, and displays a result or the like of the processing on a display screen or the like of output display section 19 as appropriate.

Control section 11 includes a central processing unit (CPU), a hard disk drive (HDD), and a random access memory (RAM), for example. The CPU reads various types of programs stored in the HDD and loads the programs onto the RAM and integrally control operations of the sections of ultrasound diagnostic apparatus U in accordance with the programs. The HDD stores therein control programs for operating ultrasound diagnostic apparatus U and various types of processing programs, and various types of setting data and/or the like. These programs and setting data may be stored in an auxiliary storage using a nonvolatile memory such as a flash memory including a solid state drive (SSD), for example, in a readable, writable, and updatable manner. The RAM is a volatile memory such as an SRAM or DRAM and provides the CPU with a working memory space and stores therein temporary data.

Control section 11 includes switching control section 111. Switching control section 111 configures settings to deflect the traveling direction of ultrasound by first, second, and third transmission and reception sections 211, 212, and 213 (see FIG. 3) arranged in the short-axis direction, when the leading end of puncturing needle 3 deflects in a direction orthogonal to a scanning direction by transmission and reception section arrangement 21 and is outside of a capturing range, based on the position information of puncturing needle 3 identified in image processing section 16. Switching control section 111 then outputs a control signal according to the settings. The operation of switching control section 111 may be executed by means of software using the CPU and/or RAM of control section 11.

Transmission driving section 12 outputs a pulse signal to be supplied to ultrasound probe 2 in accordance with the control signal inputted from control section 11 to cause ultrasound probe 2 to send ultrasound. Transmission driving section 12 includes, for example, a clock generation circuit, a pulse width setting section, a pulse generation circuit, and a delay circuit. The clock generation circuit is a circuit for generating a clock signal that determines transmission timing and/or transmission frequency of a pulse signal. The pulse width setting section sets a waveform (shape), a voltage magnitude, and a pulse width of a transmission pulse to be outputted from the pulse generation circuit. The pulse generation circuit generates a transmission pulse based on the settings of the pulse width setting section and outputs the transmission pulse to interconnection paths different for respective transmission and reception sections 210 of ultrasound probe 2. The delay circuit counts clock signals outputted from the clock generation circuit, and when a set delay period passes, the delay circuit causes the pulse width generation circuit to generate and output a transmission pulse to each of the interconnection paths.

Reception processing section 13 is a circuit for acquiring a received signal inputted from ultrasound probe 2 in accordance with a control made by control section 11. Reception processing section 13 includes, for example, an amplifier, an A/D converter circuit, and a phasing and addition circuit. The amplifier is a circuit for amplifying, using a predetermined amplification factor, the received signal in accordance with ultrasound received by each transmission and reception section 210 of ultrasound probe 2. The A/D converter circuit is a circuit for converting the amplified received signal into digital data with a predetermined sampling frequency. The phasing and addition circuit is a circuit for generating sound ray data by adjusting a time phase by providing a delay time to each of the interconnection paths corresponding to transmission and reception sections 210, respectively, and adding the results (phasing addition).

Transmission and reception switching section 14 causes a drive signal to be transmitted from transmission driving section 12 to transmission and reception section 210 when ultrasound is emitted (transmitted) from transmission and reception 210, based on a control of control section 11, and when a signal of the ultrasound emitted from transmission and reception 210 is acquired, transmission and reception switching section 14 performs a switching operation for causing reception processing section 13 to output a received signal. Transmission driving section 12, reception processing section 13, and transmission and reception switching section 14 constitute a transmission and reception processing section.

Image generation section 15 generates a diagnostic image based on the received data of ultrasound. Image generation section 15 acquires a signal by detecting (envelope detection) the sound ray data inputted from reception processing section 13, and performs as appropriate logarithm amplification, filtering (e.g., low-pass filtering or smoothing) and/or enhancement processing. Image generation section 15 generates, as one diagnostic image, each frame image (diagnostic image) data according to B-mode displaying representing a two-dimensional structure within a cross section containing a transmission direction (depth direction of test subject) of a signal and a scanning direction of ultrasound transmitted by ultrasound probe 2, with a brightness signal in accordance with the signal strength. At this time, image generation section 15 can perform dynamic range adjustment and/or gamma correction related to displaying. Image generation section 15 may be configured to include a CPU and/or a RAM exclusively used for generating these images. Alternatively, image generation section 15 may be provided in such a way that a hardware configuration dedicated to generation of images is formed on a substrate (such as an application-specific integrated circuit (ASIC)) or is formed using a field programmable gate array (FPGA). Alternatively, image generation section 15 may be configured to perform processing related to generation of images by the CPU and RAM of control section 11.

Image processing section 16 includes storage section 161 and puncturing needle identifying section 162, for example.

Storage section 161 stores, in frame units, diagnostic image data (frame image data) processed in image generation section 15 and used in real-time displaying and/or displaying equivalent to this for a predetermined number of nearest frames. Storage section 161 is, for example, a volatile memory such as dynamic random access memory (DRAM). Alternatively, storage section 161 may be various types of high-speed rewritable nonvolatile memory. The diagnostic image data stored in storage section 161 is read in accordance with a control of control section 11 and transmitted to output displaying section 19 or outputted to outside of ultrasound diagnostic apparatus U via a communication section (illustration of this section is omitted). At this time, when the display system of output display section 19 is a television system, a digital signal converter (DSC) is provided between storage section 161 and output display section 19, and the data may be transmitted after conversion of a scanning format.

Puncturing needle identifying section 162 generates image data for identifying the position of puncturing needle 3 and applies appropriate processing to the image data to identify the position of the leading end portion of puncturing needle 3.

As a method of identifying the position of puncturing needle 3, for example, the leading end (leading end portion) of puncturing needle 3 in motion can be detected by taking a difference or correlation between a plurality of diagnostic images generated with predetermined time intervals, for example.

Operation input section 18 includes a push button switch, keyboard, mouse, or trackball, or a combination of these devices, and converts a user input operation into an operation signal and inputs the signal into ultrasound diagnostic apparatus body 1.

Output display section 19 includes a display screen using one of various display systems including liquid crystal display (LCD), organic electro-luminescent (EL) display, non-organic EL display, plasma display, and cathode ray tube (CRT) display systems, and a driving section for the display screen. Output display section 19 generates a drive signal for the display screen (display elements) in accordance with the image data generated by image processing section 16 and displays, on the display screen, a menu and/or status on the ultrasound diagnostic, and/or measurement data based on the received ultrasound. In addition, output display section 19 may be configured to display whether power supply is ON or OFF using an LED light or the like additionally provided thereto.

These operation input section 18 and output display section 19 may be integrally provided with a chassis of ultrasound diagnostic apparatus body 1, or may be attached externally via an RGB cable, USB cable, or HDMI (registered trademark) cable or the like. Alternatively, when ultrasound diagnostic apparatus body 1 is provided with an operation input terminal and display output terminal, traditional operation and display peripherals may be connected to these terminals for use.

Ultrasound probe 2 functions as an acoustic sensor that oscillates to produce ultrasound (about 1 to 30 MHz in this case) and emits the ultrasound to a test subject such as a living body, and receives a reflected wave (echo) reflected by a test subject among the emitted ultrasound and converts the reflected wave into an electric signal. Ultrasound probe 2 includes: transmission and reception section arrangement 21, which is an arrangement of three transmission and reception sections 210 for transmitting and receiving ultrasound; a plurality of switch sections 23 respectively corresponding to transmission and reception sections 210; switching configuration section 24; and operation input section 28, for example. Note that, although ultrasound probe 2 is configured to emit ultrasound to inside of a test subject from outside (surface) of ultrasound probe 2 and to receive a reflected wave of the ultrasound, one that has a shape or size to be used while being inserted into a digestive tract or a blood vessel or a body cavity or the like is included in ultrasound probe 2. Users are to operate ultrasound diagnostic apparatus U by causing a transmission and reception surface of ultrasound in ultrasound probe 2, i.e., a surface in a direction in which ultrasound is emitted from transmission and reception section arrangement 21 to be into contact with the test subject to perform ultrasound diagnostic.

Transmission and reception section arrangement 21 is an arrangement of a plurality of transmission and reception sections 210 including a piezoelectric element having electrodes provided at both ends where an electrical charge appears due a piezoelectric body and deformation (expansion and contraction) thereof.

FIG. 3 is a diagram illustrating an example of transmission and reception arrangement section 21 in ultrasound probe 2 of the present embodiment.

A direction orthogonal to a scanning direction may be referred to as a short-axis direction (corresponding to “first direction” in this invention) while the scanning direction may be referred to as a long-axis direction, and a direction orthogonal to a width direction and the long-axis direction may be referred to as a depth direction, hereinafter. In addition, a distance in a depth direction from an ultrasound transmission and reception surface may be referred to as “depth,” while a distance to a focal position from the ultrasound transmission and reception surface may be referred to as “focal point distance,” hereinafter. Note that, the term “focal position” refers to a position where ultrasound beams are focused in the short-axis direction by acoustic lens 22, hereinafter.

In ultrasound diagnostic apparatus U of this embodiment, transmission and reception section arrangement 21 is a plurality of transmission and reception sections 210 arranged in a matrix in two-dimensional plane (does not have to be a flat surface) defined by a predetermined direction (scanning direction) and a width direction (first direction) orthogonal to this scanning direction. Normally, the number of transmission and reception sections 210 arranged in the scanning direction is greater than the number of transmission and reception sections 210 arranged in the width direction. Thus, the scanning direction serves as the long-axis direction and the width direction serves as the short-axis direction. In the short-axis direction, first, second, and third transmission and reception sections 211, 212, and 213 are arranged in sequence. A set of first, second, and third transmission and reception sections 211, 212, and 213 in the short-axis direction is also referred to as a transmission and reception section set, hereinafter.

Supplying each set of a predetermined number of transmission and reception sections with a voltage pulse in sequence (including a case where a partially overlapping portion occurs) in the scanning direction in a plurality of transmission and reception sections 210 causes each piezoelectric body of transmission and reception section 210 supplied with the voltage pulse to deform (expand and contract) in accordance with an electric field generated in the piezoelectric body, and thus, ultrasound is sent. The ultrasound thus sent is emitted to positions and directions in accordance with positions and directions of transmission and reception sections 210 included in the predetermined number of transmission and reception section sets supplied with the voltage pulse, a focus direction of the sent ultrasound, and a size of shift in timing (delay). Moreover, when the ultrasound of a predetermined frequency band enters transmission and reception sections 210, the sound pressure of the ultrasound causes the thickness of the piezoelectric body to fluctuate (vibrate), so that an electrical charge in accordance with the amount of fluctuation is generated, and the electrical charge is converted into an electric signal in accordance with the amount of electrical charge and then outputted.

Switch sections 23 are provided correspondingly to transmission and reception sections 210. First, second, and third switch sections 231, 232, and 233 are provided as switch sections 23 correspondingly to first, second, and third transmission and reception sections 211, 212, and 213.

Switch section 23 switches between operation and non-operation of transmission and reception section 210 based on a switch selection signal from switching configuration section 24. The expression “operation of transmission and reception section 210” herein refers to an operation of a case where transmission and reception section 210 is selected as a driving transmission and reception section. Meanwhile, the expression “non-operation of transmission and reception section 210” herein refers to an operation of a case where transmission and reception section 210 is not selected as a driving transmission and reception section and includes a case where transmission and reception section 210 is in a second operation state (second embodiment to be described, hereinafter).

Switching configuration section 24 deflects the traveling direction of ultrasound by selecting the driving transmission and reception section to perform transmission and reception of ultrasound from among a plurality of transmission and reception sections 210 and switches the focal position of an ultrasound beam between a shallow portion and a deep portion. In ultrasound diagnostic apparatus U of the present embodiment, the traveling direction of ultrasound can be set for each transmission and reception section set as will be described, hereinafter.

Operation input section 28 receives an input operation by an operator and causes an operation in accordance with content of the operation to be performed. For example, setting of switching configuration section 24 can be manually changed in accordance with an operation performed on operation input section 28.

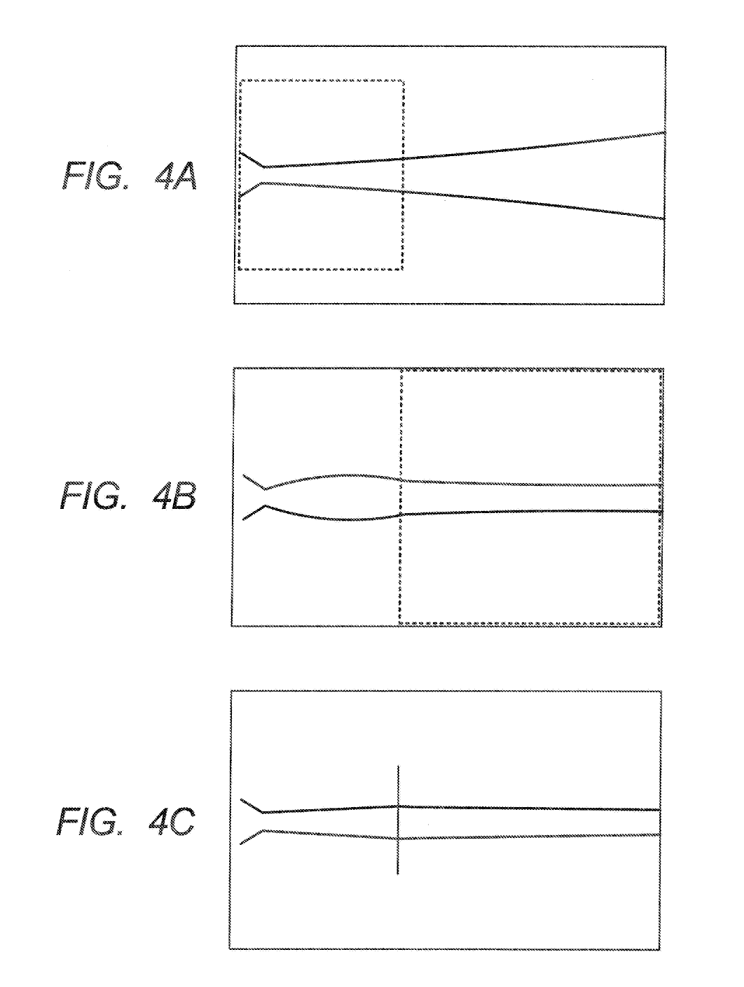

In a general 1.25D probe, transmission and reception sections 210 are split in the short-axis direction, and by reducing the width (short-axis opening width) of transmission and reception sections 210 used for transmission and reception, ultrasound beams are focused at a relatively shallow region, and by increasing the width of transmission and reception sections 210 used for transmission and reception, ultrasound beams are focused at a relatively deep region. This probe is advantageous in that ultrasound beams can be focused at the shallow and deep portions as compared with ultrasound probe 2 in which transmission and reception sections 210 are not split in the short-axis direction. In a general 1.25D probe, when a short-axis opening width is made small or large, the center of the opening is to match the center of the short-axis width. Favorable depths (portions each surrounded by a dotted rectangle) of the short-axis opening widths of the shape of an ultrasound beam when the short-axis opening width is made small as illustrated in FIG. 4A and of the shape of an ultrasound beam when the short-axis opening width is made large as illustrated in FIG. 4B are combined to form an image as a single ultrasound beam illustrated in FIG. 4C.

A ratio of the short-axis opening (short-axis split ratio) in a general 1.25D probe is preferably a split ratio of 1:2:1 in extent in terms of beam formation. Note that, “short-axis split ratio” does not necessarily refer to an accurate numerical value and includes a result of rounding an actual measurement value to an integer (approximation).

In this embodiment, in addition to a general 1.25D probe, which uses a short-axis opening while switching between short-axis openings, in a case where a probe is used while a short-axis opening is made smaller, the opening center is set not to match the center of a short-axis width, and an ultrasound beam is to be deflected.

As for the short-axis split ratio of the case where a beam is deflected and used, for example, setting an equal width (including substantially equal width), 1:1:1 for a three-way split brings advantages in terms of beam deflection angles and beam focusing properties.

In the present invention, a 1.25D probe split using a short-axis split ratio is used in the following two ways in the same probe: 1) usage in which a general short-axis opening width is switched; and 2) usage in which an ultrasound beam is deflected.

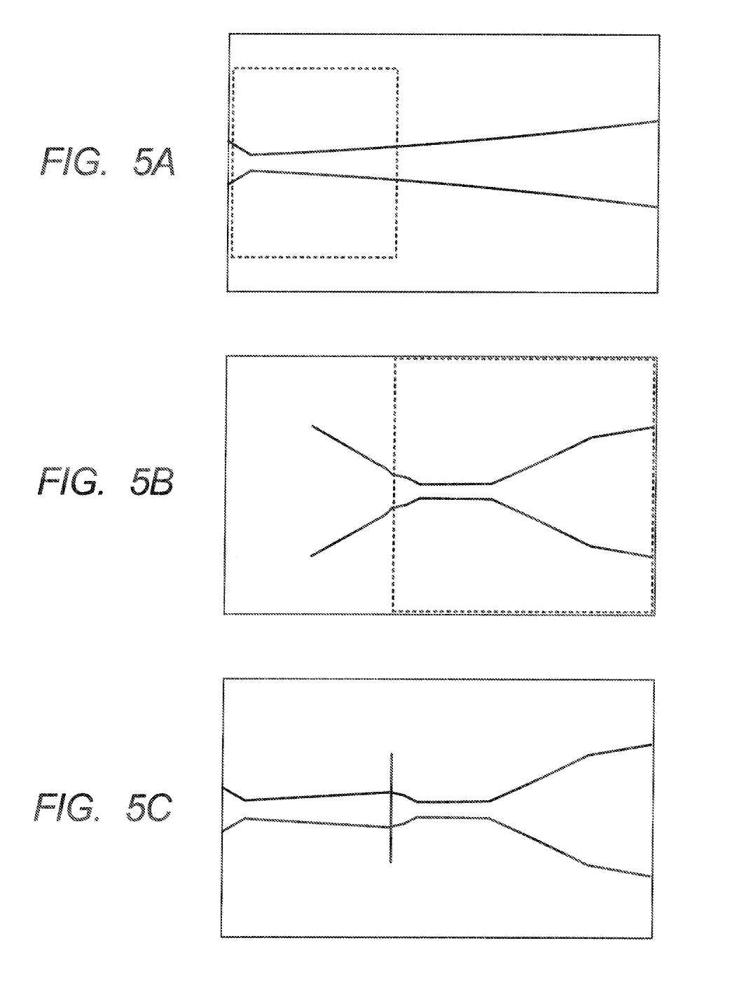

However, with the short-axis split ratio (1:1:1) suitable for a deflection beam, when the short-axis opening is wide as illustrated in FIGS. 5A to 5C, ultrasound beams are focused only at a certain depth position and spread further at a deep portion. As a result, a focal depth (length of the portion where the beam becomes thin) becomes short, and proportional beam formation cannot be obtained. Note that, this case is where lenses corresponding to respective transmission and reception sections 210 split with the short-axis split ratio (1:1:1) are all spherical.

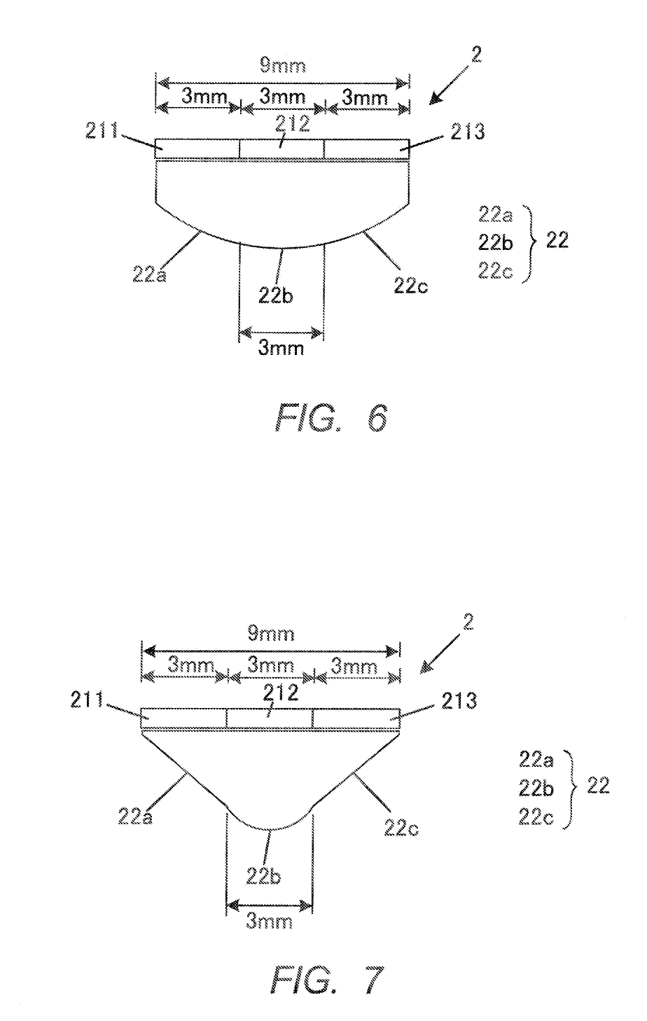

FIG. 6 illustrates an example in which the entire surface where ultrasound is transmitted and received in each transmission and reception section 210 split with a short-axis split ratio (1:1:1) and a lens corresponding to each of these sections are spherical in an ultrasound probe according to a comparison example. Transmission and reception section 210 positioned at the center in the short-axis direction is called second transmission and reception section 212, and transmission and reception sections 210 positioned symmetrically about second transmission and reception section 212 respectively at both sides of second transmission and reception section 212 are called first and third transmission and reception sections 211 and 213. Note that, the term “symmetrically” means that positions and sizes are symmetrical.

The ultrasound probe illustrated in FIG. 6 adopts a short-axis split ratio which is advantageous in deflecting a beam, but since the lens is spherical, a beam which is proportional from a shallow portion to a deep portion cannot be obtained. This does not match the object of the present invention. In this embodiment, as a method of focusing ultrasound beams to be thin and proportional from the shallow portion to the deep portion, acoustic lens 22 is configured in the following manner.

Next, a description will be given of a configuration suitable for changing a focal position of ultrasound in ultrasound diagnostic apparatus U.

FIG. 7 is a diagram illustrating a cross-sectional structure along the short-axis direction of transmission and reception section arrangement 21 in ultrasound probe 2. A cross-sectional structure along cross section A-A of FIG. 3 is illustrated herein. Note that, illustrations of switch sections 23 provided correspondingly to first, second, and third transmission and reception sections 211, 212, and 213 are omitted in FIG. 7.

As illustrated in FIG. 7, in ultrasound probe 2, acoustic lens 22 having a curvature common among first, second, and third transmission and reception sections 211, 212, and 213 arranged in the short-axis direction is provided, and the traveling directions of the ultrasound of first, second, and third transmission and reception sections 211, 212, and 213 are deflected, and ultrasound beams are focused in width in the short-axis direction. In general, silicon or the like is used for acoustic lens 22. Alternatively, a material other than silicon may be selected as appropriate in accordance with a desired ultrasound refractive index.

Second lens portion 22b positioned at a center portion of acoustic lens 22 in the short-axis direction has a spherical shape having a predetermined curvature.

Furthermore, first and third lens portions 22a and 22c positioned respectively at both sides of second lens portion 22b each have an aspherical shape. The term “aspherical shape” refers to a surface that is not spherical, and includes a flat surface having a curvature equal to “0.” The shapes of first and third lens portions 22a and 22c are not limited to the shape mentioned above, and various aspherical shapes are possible. For example, in first and third lens portions 22a and 22c, a shape in which the focal position becomes deeper at a position closer to an end portion opposite to an end portion on the side of second lens portion 22b is adopted as an aspherical shape. Accordingly, an ultrasound beam is proportionally narrowed at the deep portion. In the manner described above, when first, second and third transmission and reception sections 211, 212, and 213 are selected as a driving transmission and reception section (when the short-axis width is “3”), the focal position can be deep, and ultrasound beams can be focused to be proportionally narrow at the deep portion. Note that, the entire surface where ultrasound is transmitted and received in first and third lens portions 22a and 22c is an aspherical shape. Setting the entire surface to have an aspherical shape allows the focal position to be wide from a shallow position to a deep position. Note that, as first and third lens portions 22a and 22c, the effect of setting the focal position deep can be produced as long as they partially have an aspherical shape, so that it is not necessarily to set the entire surface to have an aspherical shape. Moreover, as long as at least the shapes of first and third lens portions 22a and 22c are aspherical, the shape of second lens portion 22b may be spherical or aspherical.

In this embodiment, the curvatures of the aspherical surfaces in first and third lens portions 22a and 22c are curvatures that become closer to the curvature of second lens portion 22b as the surfaces approach second lens portion 22b from the ends thereof in the short-axis direction.

In acoustic lens 22, second lens portion 22b is provided correspondingly to second transmission and reception section 212. First lens portion 22a is provided correspondingly to first transmission and reception section 211. Moreover, third lens portion 22c is provided correspondingly to third transmission and reception section 213. As illustrated in FIG. 7, the width of each of transmission and reception sections 211, 212, and 213 in the short-axis direction is approximately 3.0 mm, so that the width of each of second lens portion 22b, and first and third lens portions 22a and 22c in the short-axis direction is approximately 3.0 [mm]. Moreover, in this embodiment, first, second, and third lens portions 22a, 22b, and 22c are provided correspondingly to first, second, and third transmission and reception sections 211, 212, and 213, but even in a case where lens portions are provided correspondingly to five or more transmission and reception sections, setting first and third lens portions 22a and 22c positioned at both sides of second lens portion 22b at the center to have an aspherical shape, the focal position of ultrasound beams can be deep, and the ultrasound beams can be focused to be proportionally thin at the deep portion.

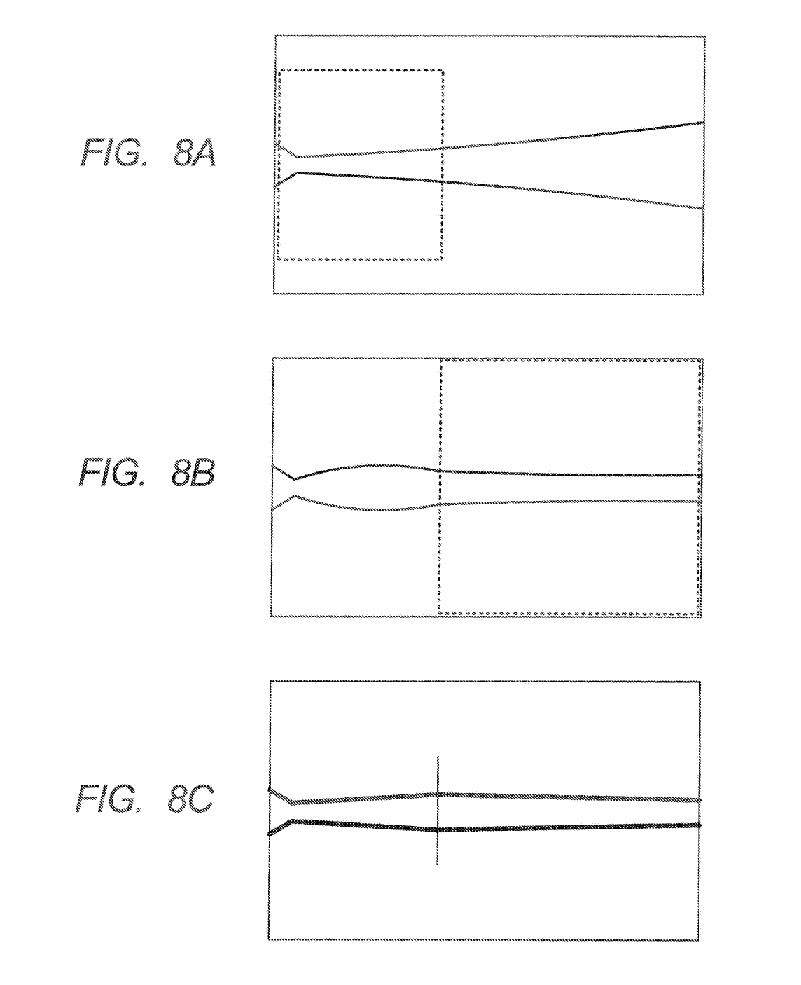

FIG. 8A is a diagram illustrating a relationship between second transmission and reception section 212 to be used and the shape of an ultrasound beam. FIG. 8B is a diagram illustrating a relationship between first, second, and third transmission and reception sections 211, 212, and 213 to be used and the shape of an ultrasound beam. FIG. 8C is a diagram illustrating a combined shape of an ultrasound beam. The term “transmission and reception section 210 to be used” refers to transmission and reception section 210 selected by switching configuration section 24 as a driving transmission and reception section.

When second transmission and reception section 212 (see FIG. 7) is selected by switching configuration section 24 as a driving transmission and reception section as illustrated in FIG. 8A, acoustic lens 22 focuses a transmission and reception beam of ultrasound from second transmission and reception section 212 such that the beam becomes thin at a focal position where a focal point distance is shallow. Moreover, when first, second, and third transmission and reception sections 211, 212, and 213 (see FIG. 7) are selected by switching configuration section 24 as a driving transmission and reception section, since first and third lens portions 22a and 22c have an aspherical shape, acoustic lens 22 can focus ultrasound beams such that the ultrasound beams become proportionally thin at a focal position where the focal point distance is deep as illustrated in FIG. 8B. In addition, by switching, using switch section 23, the transmission and reception section to be used to second transmission and reception section 212 when an ultrasound beam is focused at a shallow region, and by switching, using switch section 23, the transmission and reception section to be used to first, second, and third transmission and reception sections 211, 212, and 213 when ultrasound beams are focused at a deep region, as illustrated in FIG. 8C, ultrasound beams can be substantially focused to be proportionally thin over a wide range from the shallow portion to the deep portion.

As has been described above, ultrasound probe 2 according the first embodiment includes: a plurality of transmission and reception sections 210 arranged in the short-axis direction; acoustic lens 22 configured to focus transmission and reception beams of ultrasound in the short-axis direction; switching configuration section 24 configured to select a driving transmission and reception section from among the plurality of transmission and reception sections 210; and switch section 23 configured to switch an operation of transmission and reception section 210 based on a switch selection signal of switching configuration section 24. Acoustic lens 22 includes first, second, and third lens portions 22a, 22b, and 22c correspondingly to first, second, and third transmission and reception sections 211, 212, and 213, and first and third lens portions 22a and 22c each have an aspherical shape.

As has been described above, selecting a driving transmission and reception section by switching configuration section 24 from among a plurality of transmission and reception sections 210 allows for changing the focal position of an ultrasound beam, thereby making it possible to easily change a capturing range. Thus, an ultrasound probe with good usability and a simple configuration can be provided. In addition, an electronic circuit or the like is not required, and the number of electrodes to be extracted can be small, so that no complex configuration is required, resulting in low costs. Moreover, the following functions can be achieved by single ultrasound probe 2: improving the spatial resolution by focusing an ultrasound beam to be proportionally thin in a wide range from a shallow portion to a deep portion; and keeping puncturing needle 3 within an ultrasound beam whose traveling direction is sufficiently deflected even when puncturing needle 3 is shifted in the short-axis direction.

### Second Embodiment

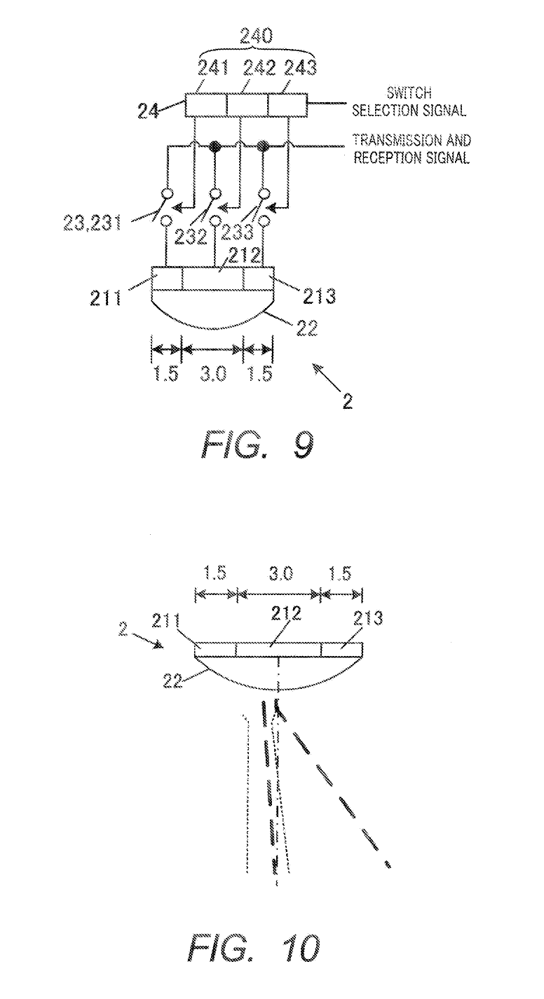

FIG. 9 is a diagram illustrating ultrasound probe 2 according to a comparison example. In FIG. 9, ultrasound probe 2 in general has a short-axis split ratio of 1:2:1 is illustrated. In this case, an opening width when second transmission and reception section 212 is selected by switching configuration section 24 as a driving transmission and reception section is, for example, 3 [mm] (small opening). In addition, an opening width when first, second, and third transmission and reception sections 211, 212, and 213 are selected by switching configuration section 24 as a driving transmission and reception section is, for example, 6 [mm] (large opening).

The short-axis split ratio of 1:2:1 is a preferable short-axis split ratio for focusing ultrasound beams to be proportionally thin from a shallow portion to a deep portion as described above.

FIG. 10 is a diagram illustrating a relationship between transmission and reception section 210 to be used and a shape of an ultrasound beam. In FIG. 10, the shape of an ultrasound beam of a case where only first transmission and reception section 211 is used is indicated by bold broken lines while the shape of an ultrasound beam when first and second transmission and reception sections 211 and 212 are used is indicated by dotted lines.

As the shape of the ultrasound beam is indicated by bold broken lines in FIG. 10, when only first transmission and reception section 211 is selected, the opening width to be used is so small compared to the entire opening width that the ultrasound beam shape does not become thin, and because the directivity of the ultrasound at a shallow portion is on a side opposite to an intended side due to deflection, the position of puncturing needle 3 may be wrongly recognized. Meanwhile, as the shape of the ultrasound beam is indicated by dotted lines in FIG. 10, when first and second transmission and reception sections 211 and 212 are selected, since third transmission and reception section 213 which is not selected is small, the deflection of the traveling direction of the ultrasound is also small, and there is no difference compared to the traveling direction of the ultrasound in a case where first, second, and third transmission and reception sections 211, 212, and 213 are selected.

When puncturing needle 3 is to be inserted toward a specific region in a test subject, a position movement (shift) of puncturing needle 3 needs to be checked successively. When the position moment of puncturing needle 3 is relatively large, the traveling direction of the ultrasound needs to be significantly deflected, so that ultrasound probe 2 in general having a short-axis split ratio of 1:2:1, which involves a small deflection of the traveling direction of ultrasound, is unsuitable for identifying a position movement of puncturing needle 3.

In the second embodiment, in order to make ultrasound probe 2 suitable for deflecting the traveling direction of ultrasound while the short-axis split ratio is set to 1:2:1, second switch section 232 includes switching element 31 and electric circuit 32 connected in parallel with switching element 31.

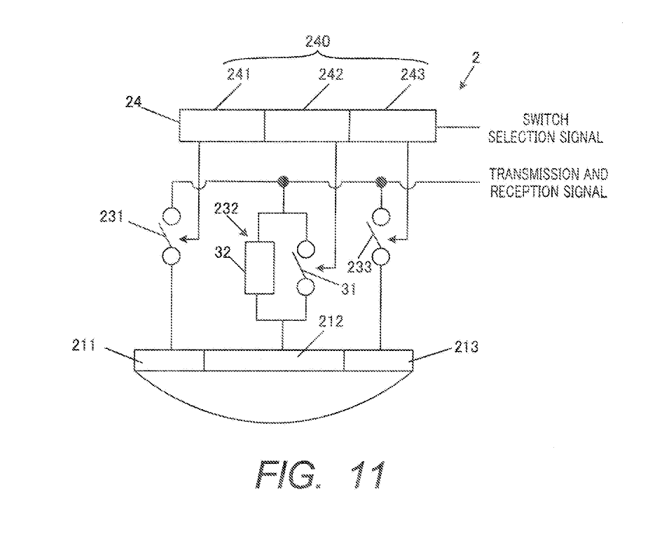

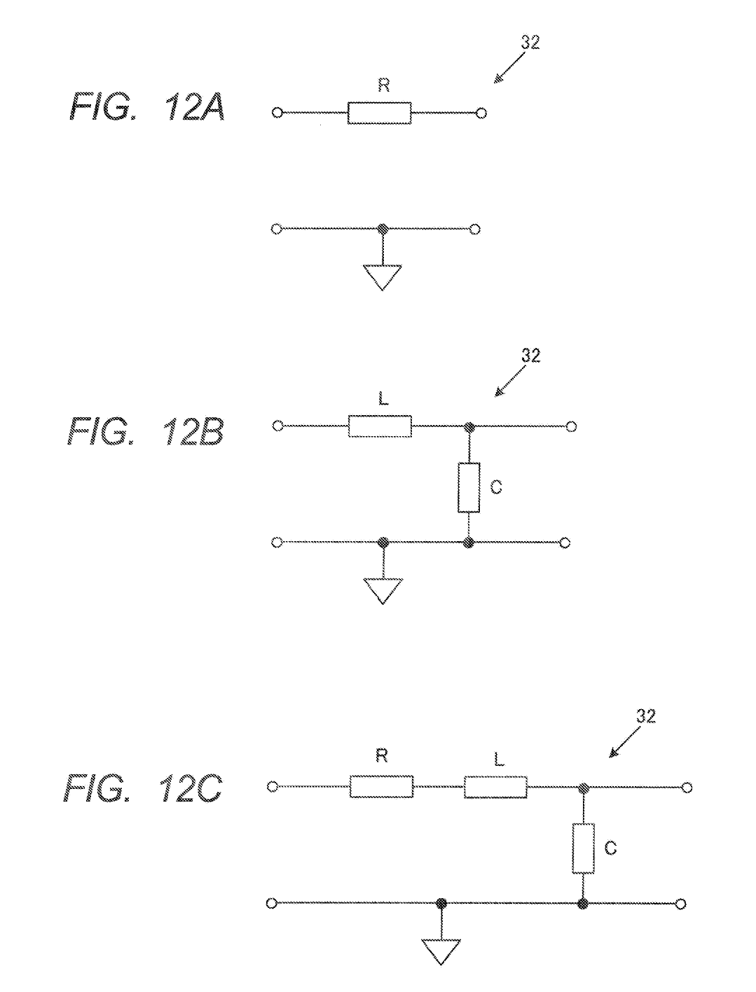

FIG. 11 is a diagram illustrating ultrasound probe 2 according to the second embodiment. FIGS. 12A, 12B, and 12C are each a diagram illustrating a configuration of electric circuit 32.

As illustrated in FIG. 11, first, second, and third transmission and reception sections 211, 212, and 213 transmit and receive transmission and reception signals via first, second, and third switch sections 231, 232, and 233, respectively. Switching configuration section 24 includes register 240. Registers 241, 242, and 243 are provided correspondingly to first, second, and third switch sections 231, 232, and 233. First, second, and third switch sections 231, 232, and 233 are turned ON and OFF in accordance with switch selection signals stored in registers 241, 242, and 243, which are previously inputted from control section 11. First, second, and third switch sections 231, 232, and 233 are not limited in particular, but a field-effect transistor (FET), for example, is preferably used taking into consideration power consumption and/or breakdown voltage performance related to ultrasound transmission and reception.

Second switch section 232 includes switching element 31 and electric circuit 32 to be connected in parallel with switching element 31. Thus, when second transmission and reception section 212 is selected by switching configuration section 24 as a driving transmission and reception section, second transmission and reception section 212 transmits and receives ultrasound not via electric circuit 32 by turning ON switching element 31. This case is referred to as a first operation state of second transmission and reception section 212. When the traveling direction of ultrasound is deflected, second transmission and reception section 212 is not selected by switching configuration section 24 as a driving transmission and reception section, and switching element 31 is turned OFF, so that second transmission and reception section 212 transmits and receives ultrasound via electric circuit 32. This case is referred to a second operation state of second transmission and reception section 212 (corresponding to “non-operation of transmission and reception section” of this invention).

For example, when the traveling direction of ultrasound is to be deflected rightward in FIG. 11, switching element 31 is turned OFF, and first switch section 231 is turned ON. Thus, second transmission and reception section 212 transmits and receives ultrasound via electric circuit 32 (second operation state). Moreover, first transmission and reception section 211 is switched to a driving transmission and reception section. Meanwhile, when the traveling direction of ultrasound is to be deflected leftward in FIG. 11, switching element 31 is turned OFF, and third switch section 233 is turned ON. Thus, second transmission and reception section 212 transmits and receives ultrasound via electric circuit 32 (second operation state). Moreover, third transmission and reception section 213 is switched to a driving transmission and reception section.

When only second transmission and reception section 212 is used (small opening), switching element 31 is turned ON, and first and third switch sections 231 and 233 are turned OFF. Thus, switching is made such that second transmission and reception section 212 transmits and receives ultrasound not via electric circuit 32 (first operation state). When first, second, and third transmission and reception sections 211, 212, and 213 are used (large opening), switching element 31 is turned ON. Thus, switching is made such that second transmission and reception section 212 transmits and receives ultrasound not via electric circuit 32 (first operation state). In addition, first and third switch sections 231 and 233 are turned ON. Thus, first and third transmission and reception sections 211 and 213 are switched to a driving transmission and reception section.

Electric circuit 32 is composed of: a circuit composed of only resistor R (see FIG. 12A); a circuit composed of LC (see FIG. 12B); and a circuit composed of RLC (see FIG. 12C).

When switching element 31 of FIG. 11 is turned OFF, a signal to be added to second transmission and reception section 212 and an electric signal resulting from conversion of the ultrasound received by second transmission and reception section 212 pass through electric circuit 32. When switching element 31 is turned ON, the signal and the electric signal do not pass through electric circuit 32.

In a configuration without electric circuit 32, when first and second transmission and reception sections 211 and 212 are compared, second transmission and reception section 212 is longer in width in the short-axis direction and has a wide area. Thus, second transmission and reception section 212 has a higher sensitivity, and becomes dominant compared to first transmission and reception section 211, so that the deflection angle of an ultrasound beam becomes small.

In this embodiment, the sensitivity of second transmission and reception section 212 is reduced by using resistor R in electric circuit 32 as illustrated in FIG. 12A to balance between first and second transmission and reception sections 211 and 212, and thus, it is made possible to allow for a large deflection angle of an ultrasound beam.

Furthermore, a signal phase of second transmission and reception section 212 can be corrected to first transmission and reception section 211 to be shifted by using an LC circuit for electric circuit 32 as illustrated in FIG. 12B, and thus, the deflection angle of an ultrasound beam can be made larger.

Moreover, the two effects mentioned above can be brought about by using a circuit composed of LCR for electric circuit 32 as illustrated in FIG. 12C, and thus, the deflection angle of an ultrasound beam can be made larger.

### Third Embodiment

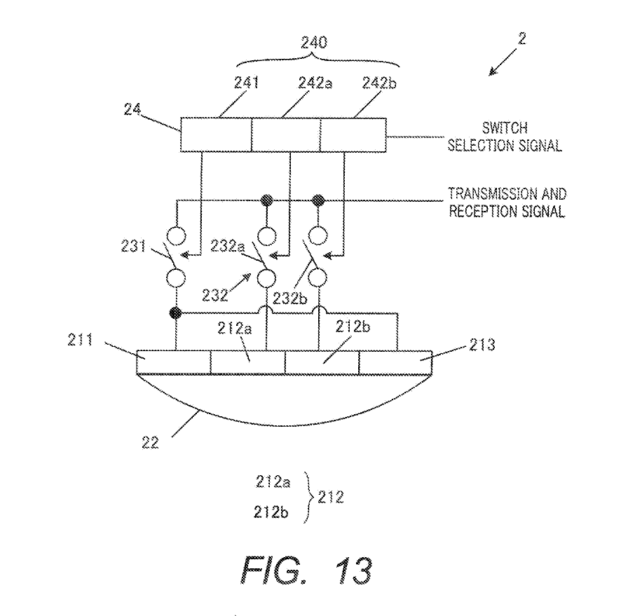

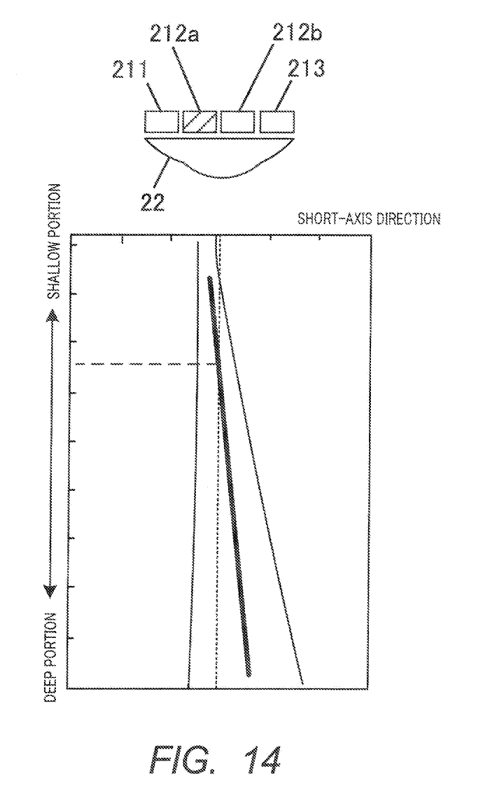

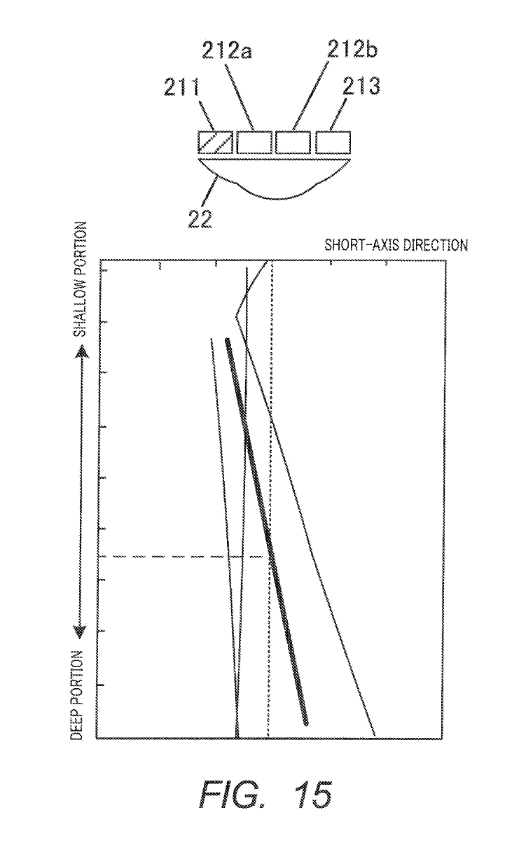

Next, ultrasound probe 2 according to a third embodiment will be described with reference to FIGS. 13 to 15. FIG. 13 is a diagram illustrating an ultrasound probe. FIG. 14 is a diagram illustrating a relationship between first section 212a to be used and the traveling direction of ultrasound. FIG. 15 is a diagram illustrating a relationship between first transmission and reception section 211 to be used and the traveling direction of ultrasound.

In ultrasound probe 2 having a short-axis split ratio of 1:2:1, when transmission and reception of ultrasound is performed by selecting, for example, only first transmission and reception section 211 (short-axis width is “1”) by switching configuration section 24, the shape of an ultrasound beam as illustrated in FIG. 15 is formed.

The depth of an intersection between the line of the traveling direction of ultrasound and the center axis of the entire short-axis direction becomes relatively deep. In other words, the directivity of ultrasound appears on a side opposite to an intended side for deflection of the traveling direction of the ultrasound at a shallow portion, so that the position of puncturing needle 3 may be wrongly recognized.

In the second embodiment, in ultrasound probe 2 having a short-axis split ratio of 1:2:1, second switch section 232 includes switching element 31 and electric circuit 32 connected in parallel with switching element 31. With this configuration, the short-axis split ratio is made functionally close to 1:1:1.

In contrast to this, in the third embodiment, in ultrasound probe 2 having a short-axis split ratio of 1:2:1, second transmission and reception section 212 is sectioned into first and second sections 212a and 212b at a center of the short-axis direction as a boundary, and by selecting one of first and third transmission and reception sections 211 and 213 and one of first and second sections 212a and 212b by switching configuration section 24 as a driving transmission and reception section, the directivity of ultrasound is made to appear on an intended side for deflection of the traveling direction of ultrasound at a shallow portion.

As illustrated in FIG. 13, first switch section 231 is connected to first and third transmission and reception sections 211 and 213. First switching element 232a is connected to first section 212a. Second switching element 232b is connected to second section 212b. Registers 241, and 242a and 242b are provided correspondingly to first switch section 231, and first and second switching elements 232a and 232b.

When only second transmission and reception section 212 is used (small opening), switching configuration section 24 turns ON first and second switching elements 232a and 232b, and turns OFF first switch section 231. Thus, first and second sections 212a and 212b are switched to a driving transmission and reception section.

When first, second, and third transmission and reception sections 211, 212, and 213 are used (large opening), switching configuration section 24 turns ON first switch section 231, and first and second switching elements 232a and 232b. Thus, first and third transmission and reception sections 211 and 213 and first and second sections 212a and 212b are switched to a driving transmission and reception section.

FIG. 14, for example, illustrates a shape of an ultrasound beam formed using first section 212a. As illustrated in FIG. 14, the directivity of ultrasound appears on an intended side for deflection of the traveling direction of ultrasound at a shallow portion. As described, a favorable deflection beam can be obtained by using this technique.

As described above, ultrasound probe 2 according to the third embodiment includes: a plurality of transmission and reception sections 210 arranged in a short-axis direction; acoustic lens 22 configured to focus transmission and reception beams of ultrasound; switching configuration section 24 configured to select a driving transmission and reception section from among the plurality of transmission and reception sections 210; and switch section 23 configured to switch an operation of transmission and reception section 210 based on a switch selection signal from switching configuration section 24. In addition, second switch section 212 includes first and second sections 212a and 212b, and switch section 23 includes first and second switch sections 231 and 232, and second switch section 232 includes first and section switching elements 232a and 232b.

In this configuration, a focal position of a transmission and reception beam is changed to a deep portion by switching first and third transmission and reception sections 211 and 213 to a driving transmission and reception section and switching first and second sections 212a and 212b of second transmission and reception section 212 to a driving transmission and reception section by selection made by switching configuration section 24, and the focal point is changed to a shallow position by not switching first and third transmission and reception sections 211 and 213 to a driving transmission and reception section, but switching first and second sections 212a and 212b of second transmission and reception section 212 to a driving transmission and reception section by selection made by switching configuration section 24.

Moreover, switching element 212a is switched to a driving transmission and reception section by selection made by switching configuration section 24, thereby favorably deflecting the traveling direction of ultrasound.

In the manner described above, focusing of ultrasound beams from the shallow portion to the deep portion with the short-axis split rate equal to 1:2:1 is made possible, and the traveling direction of ultrasound can be favorably deflected.

Note that, although acoustic lens 22 in which first and third lens portions 22a and 22c each have an aspherical shape is provided in ultrasound probe 2 having a short-axis split ratio of 1:1:1 in the embodiment described above, the present invention is not limited to this configuration. For example, acoustic lens 22 may be provided in ultrasound probe 2 having a short-axis split ratio of 1:2:1. In this case, the aspherical shapes of first and third lens section 22a and 22c may be matched to ultrasound probe 2 having a short-axis split ratio of 1:2:1.

In the embodiment described above, a description has been given of the case where a control operation for configuring settings of switching configuration section 24 is performed in ultrasound diagnostic apparatus body 1, but the present invention is not limited to this case. For example, switching configuration section 24 may include a control section (switching control section), and ultrasound probe 2 may be caused to perform a control operation related to switching of switch section 23. Moreover, instead of or in addition to the input operation to operation input section 28 of ultrasound probe 2, it is also possible to cause ultrasound probe 2 to configure settings such as switching of a deflection direction in accordance with the input operation to input operation section 18 of ultrasound diagnostic apparatus body 1. Thus, the switching operation related to deflection of a capturing range can be completed within ultrasound probe 2, so that transmission and reception of a control signal with ultrasound diagnostic apparatus body 1 is made easier. Moreover, switching configuration section 24 may be included in an ultrasound diagnostic apparatus body.

In the embodiments described above, ultrasound diagnostic apparatus U is configured to include ultrasound probe 2 and ultrasound diagnostic apparatus body 1, but ultrasound probe 2 capable of independently performing an operation and deflection control may be connected to ultrasound diagnostic apparatus body 1 in general and used.

In the embodiments described above, when only one of first, second, and third transmission and reception sections 211, 212, and 213 is to be used for transmission and reception, an S/N ratio decreases significantly as the ultrasound transmission and reception intensity decreases. For this reason, the widths and/or voltage magnitude or the like of first, second, and third transmission and reception sections 211, 212, and 213 may be configured to set an S/N ratio (received signal strength) with which puncturing needle 3 is surely detected.

Moreover, the arrangement in the scanning direction does not have to be a linear scan type, and may be another arrangement, a sector scan type, a convex type, or a radial scan type or the like.

In the embodiments described above, a description has been given of the case where puncturing needle 3 is a part of ultrasound diagnostic apparatus U, which is attached to ultrasound probe 2 by attachment section 4, but as long as puncturing needle 3 is to be inserted while being displayed in a diagnostic image, a configuration including puncturing needle 3 as a component separate from ultrasound diagnostic apparatus U may be employed.

The embodiments disclosed herein are merely exemplifications of the present invention, and the technical scope of the present invention should not be understood in a limited way by these embodiments. More specifically, the present invention may be implemented in various forms without departing from the gist or primary features of the present invention.

What is claimed is:

1. An ultrasound probe comprising:
a plurality of transmission and reception sections arranged along a predetermined first direction and configured to transmit ultrasound to a test subject and receive a reflection wave of the ultrasound;
an acoustic lens configured to focus, in the first direction, ultrasound beams transmitted and received by the transmission and reception sections; and
a switch section configured to switch between operation and non-operation of the transmission and reception sections, wherein:
the transmission and reception sections include first, second, and third transmission and reception sections, the second transmission and reception section being centrally positioned, the first and the third transmission and reception sections being disposed symmetrically at both sides of the second transmission and reception section,
the acoustic lens includes first, second, and third lens portions respectively corresponding to the first, the second, and the third transmission and reception sections,
the switch section causes, when a traveling direction of the ultrasound is straight ahead, the second transmission and reception section alone or all of the first, the second, and the third transmission and reception sections to operate, and causes, when the traveling direction of the ultrasound is to be deflected, the first or the third transmission and reception section to operate, and
the first and the third lens portions have an aspherical shape.

2. The ultrasound probe according to claim 1, wherein a split ratio of the first, the second, and the third transmission and reception sections in the first direction is 1:1:1.

3. The ultrasound probe according to claim 1, wherein, in the first and the third lens portions, a surface where the ultrasound is transmitted and received is entirely aspherical.

4. The ultrasound probe according to claim 1, wherein a curvature of the aspherical shape becomes closer to a curvature of the second lens portion as the aspherical shape extends to the second lens portion from each end of the aspherical shape in the first direction of the first and the third lens portions.

5. An ultrasound probe comprising:
a plurality of transmission and reception sections arranged along a predetermined first direction and configured to transmit ultrasound to a test subject and receive a reflection wave of the ultrasound;
an acoustic lens configured to focus, in the first direction, ultrasound beams transmitted and received by the transmission and reception sections; and
a switch section configured to switch between operation and non-operation of the transmission and reception sections, wherein:
the transmission and reception sections include first, second, and third transmission and reception sections, the second transmission and reception section being centrally positioned, the first and the third transmission and reception sections being disposed symmetrically at both sides of the second transmission and reception section, and
the switch section includes first, second, and third switch sections respectively corresponding to the first, the second, and the third transmission and reception sections, wherein
the second switch section includes a switching element and an electric circuit connected in parallel with the switching element, and causes the second transmission and reception section to operate via the electric circuit or not via the electric circuit by the switching element.

6. The ultrasound probe according to claim 5, wherein a split ratio of the first, the second, and the third transmission and reception sections in the first direction is 1:2:1.

7. The ultrasound probe according to claim 5, wherein the electric circuit reduces sensitivity of the second transmission and reception section of a case where the second transmission and reception section is caused to operate via the electric circuit compared to a case where the second transmission and reception section is caused to operate not via the electric circuit.

8. The ultrasound probe according to claim 5, wherein the electric circuit includes a phase shift circuit.

9. An ultrasound probe comprising:
a plurality of transmission and reception sections arranged along a predetermined first direction and configured to transmit ultrasound to a test subject and receive a reflection wave of the ultrasound;
an acoustic lens configured to focus, in the first direction, ultrasound beams transmitted and received by the transmission and reception sections; and
a switch section configured to switch between operation and non-operation of the transmission and reception sections, wherein
the transmission and reception sections include first, second, and third transmission and reception sections, the second transmission and reception section being centrally positioned, the first and the third transmission and reception sections being disposed symmetrically at both sides of the second transmission and reception section, wherein
the second transmission and reception section includes first and second sections obtained by centrally splitting the second transmission and reception section, and
the switch section includes switching elements corresponding to the first and the second sections, wherein
the switch section causes, when a traveling direction of the ultrasound is straight ahead, the second transmission and reception section alone or all of the first, the second, and the third transmission and reception sections to drive, and causes, when the traveling direction of the ultrasound is to be deflected, one of the first and the second sections of the second transmission and reception section to drive by the corresponding switching element.

10. The ultrasound probe according to claim 9, wherein a split ratio of the first, the second, and the third transmission and reception sections in the first direction is 1:2:1.

11. The ultrasound probe according to claim 9, wherein the switch section includes a first switch section connected in common to the first and the third transmission and reception sections.

12. An ultrasound diagnostic apparatus comprising:
the ultrasound probe according to claim 1; and
a transmission and reception processing section configured to perform a transmission and reception operation of ultrasound in the ultrasound probe.

13. An ultrasound diagnostic apparatus comprising:
the ultrasound probe according to claim 5; and
a transmission and reception processing section configured to perform a transmission and reception operation of ultrasound in the ultrasound probe.

14. An ultrasound diagnostic apparatus comprising:
the ultrasound probe according to claim 9; and
a transmission and reception processing section configured to perform a transmission and reception operation of ultrasound in the ultrasound probe.
